(19) United States
(12) Patent Application Publication
(10) Pub. No.: US 2021/0287761 A1
(43) Pub. Date: Sep. 16, 2021

(54) SYMMETRY-BASED QUANTUM COMPUTATIONAL CHEMISTRY

(71) Applicant: River Lane Research Ltd., Cambridge (GB)

(72) Inventors: Joan Camps, Cambridge (GB); Dan Underwood, Cambridge (GB); Ophelia Crawford Clarke, Cambridge (GB)

(73) Assignee: River Lane Research Ltd., Cambridge (GB)

(21) Appl. No.: 17/249,761

(22) Filed: Mar. 11, 2021

Related U.S. Application Data
(60) Provisional application No. 62/988,681, filed on Mar. 12, 2020.

(51) Int. Cl. G16C 10/00; G06N 10/00; G06F 15/16

(52) U.S. Cl. CPC G16C 10/00; G06F 15/16; G06N 10/00

(57) ABSTRACT

A computing system can be configured to determine a compressed quantum circuit architecture, for a quantum computer, based on a point-symmetry group of a physical system. The computing system can comprise a classical computer operatively coupled to the quantum computer. The classical computer can be configured to transmit a symmetrized-unitary operator to the quantum computer to enable configuration of the compressed quantum circuit architecture and application of the compressed quantum circuit architecture to a quantum memory containing a first quantum basis state of the physical system stored in a plurality of qubits. The first quantum basis state transforms according to a first irreducible representation of the point-symmetry group.

### CROSS-REFERENCE TO RELATED APPLICATIONS

This application claims priority to U.S. Provisional Application No. 62/988,681, filed Mar. 12, 2020, titled SYMMETRY-BASED QUANTUM COMPUTATIONAL CHEMISTRY, which is incorporated herein by reference in its entirety.

### BACKGROUND

The present disclosure relates to apparatus, systems and methods for configuring quantum circuitry within a quantum computer, in particular, although not necessarily, for performing quantum computational chemistry.

### SUMMARY

According to a first aspect of the present disclosure there is provided a computing system configured to determine a compressed quantum circuit architecture, for a quantum computer, based on a point-symmetry group of a physical system. The computing system comprises a classical computer operatively coupled to the quantum computer. The classical computer can receive the point-symmetry group, wherein the point-symmetry group comprises a plurality of elements, each element corresponding to a symmetry operation on all quantum basis states of the physical system. The classical computer can receive a unitary operator based on a plurality of parameters. The unitary operator can encode a quantum circuit architecture. The classical computer can determine a symmetrized-unitary operator based on the unitary operator. The symmetrized-unitary operator: transforms as the identity representation of the point-symmetry group; is based on a proper subset only of the plurality of parameters; and encodes the compressed quantum circuit architecture. The classical computer can transmit the symmetrized-unitary operator to the quantum computer to enable configuration of the compressed quantum circuit architecture and application of the compressed quantum circuit architecture to a quantum memory containing a first quantum basis state of the physical system stored in a plurality of qubits. The first quantum basis state transforms according to a first irreducible representation of the point-symmetry group.

Optionally, the computing system may comprise the quantum computer. The quantum computer can: prepare the first quantum basis state in the quantum memory; receive the symmetrized-unitary operator; prepare a compressed quantum circuit based on the symmetrized-unitary operator; apply the compressed quantum circuit to the quantum memory; determine a first plurality of qubit measurement values for the first quantum basis state; and transmit the first plurality of qubit measurement values to the classical computer.

Optionally, the quantum computer may prepare a second quantum basis state in the quantum memory. The second quantum basis state transforms according to a second irreducible representation of the point-symmetry group different to the first irreducible representation. The quantum computer may: apply the compressed quantum circuit to the quantum memory; determine a second plurality of qubit measurement values for the second quantum basis state; and transmit the second plurality of qubit measurement values to the classical computer.

Optionally, the classical computer may estimate an expectation of a quantum mechanical operator, for the physical system, based on the first plurality of qubit measurement values.

Optionally, the classical computer may vary one or more of the plurality of parameters and estimate an optimized-eigenvalue of the quantum mechanical operator by successively controlling the quantum computer to prepare one or more varied compressed quantum circuits and apply each in turn of the one or more varied compressed quantum circuits to the quantum memory containing each in turn of one or more of the quantum basis states of the physical system.

Optionally, the quantum mechanical operator may be a Hamiltonian operator.

Optionally, the symmetrized-unitary operator may be determined by averaging the unitary operator over the plurality of elements of the point-symmetry group.

Optionally, the unitary operator may be an exponential of an anti-Hermitian operator, and the symmetrized-unitary operator may be determined by exponentiating a symmetrized-anti-Hermitian operator determined by averaging the anti-Hermitian operator over the plurality of elements of the point-symmetry group.

Optionally, the computing system may determine a reduction in activation energy in catalyst development. The physical system may be a molecular system. The classical computer may vary one or more of the plurality of parameters and estimate an optimized-eigenvalue of a Hamiltonian operator of the molecular system by successively controlling the quantum computer to prepare one or more varied compressed quantum circuits and apply each in turn of the one or more varied compressed quantum circuits to the quantum memory containing each in turn of one or more of the quantum basis states of the physical system.

Optionally the computing system may simulate protein-molecule interactions. The physical system may be a molecular system. The classical computer may vary one or more of the plurality of parameters and estimate an optimized-eigenvalue of a Hamiltonian operator of the molecular system by successively controlling the quantum computer to prepare one or more varied compressed quantum circuits and apply each in turn of the one or more varied compressed quantum circuits to the quantum memory containing each in turn of one or more of the quantum basis states of the physical system.

Optionally, the computing system may perform materials development. The physical system may be a unit cell of a crystalline material. The classical computer may vary one or more of the plurality of parameters and estimate an optimized-eigenvalue of a Hamiltonian operator of the physical system by successively controlling the quantum computer to prepare one or more varied compressed quantum circuits and apply each in turn of the one or more varied compressed quantum circuits to the quantum memory containing each in turn of one or more of the quantum basis states of the physical system.

Optionally, the computing system may perform any one or more of catalyst development, drug discovery or materials development.

Optionally, the point-symmetry group may comprise at least one non-trivial proper rotation.

According to a further aspect of the present disclosure there is provided a computer-implemented method for determining a compressed quantum circuit architecture, for a quantum computer, based on a point-symmetry group of a physical system. The method comprises receiving the point-symmetry group, wherein the point-symmetry group comprises a plurality of elements, each element corresponding to a symmetry operation on all quantum basis states of the physical system. The method comprises receiving a unitary operator based on a plurality of parameters. The unitary operator encodes a quantum circuit architecture. The method comprises determining a symmetrized-unitary operator based on the unitary operator. The symmetrized-unitary operator: transforms as the identity representation of the point-symmetry group; is based on a proper subset only of the plurality of parameters; and encodes the compressed quantum circuit architecture. The method comprises transmitting the symmetrized-unitary operator to a quantum computer to enable configuration of the compressed quantum circuit architecture and application of the compressed quantum circuit architecture to a quantum memory containing a first quantum basis state of the physical system stored in a plurality of qubits. The first quantum basis state transforms according to a first irreducible representation of the point-symmetry group.

According to a further aspect of the present disclosure there is provided a computer program product, or a computer readable memory medium, including one or more sequences of one or more instructions which, when executed by one or more processors, cause an apparatus to at least perform the steps of any method disclosed herein.

While the disclosure is amenable to various modifications and alternative forms, specifics thereof have been shown by way of example in the drawings and will be described in detail. It should be understood, however, that other embodiments, beyond the particular embodiments described, are possible as well. All modifications, equivalents, and alternative embodiments falling within the spirit and scope of the appended claims are covered as well.

The above discussion is not intended to represent every example embodiment or every implementation within the scope of the current or future Claim sets. The Figures and Detailed Description that follow also exemplify various example embodiments. Various example embodiments may be more completely understood in consideration of the following Detailed Description in connection with the accompanying Drawings.

### BRIEF DESCRIPTION OF DRAWINGS

One or more embodiments will now be described by way of example only with reference to the accompanying drawings in which:

FIG. 1 shows an example embodiment of a computing apparatus for determining a configuration of quantum circuitry;

FIG. 2 shows an example embodiment of a distributed classical/quantum computing system;

FIG. 3 shows an example embodiment of a method for studying a spectrum of a symmetric quantum operator; and

FIG. 4 shows an example embodiment of a computer program product.

### DETAILED DESCRIPTION

Quantum chemistry is expected to be one of the main applications of the quantum computer in the NISQ era (noisy intermediate-size quantum devices). Two limiting factors in NISQ devices are (i) the size of the system to be studied, that translates into a requirement on the number of qubits needed—the size of the quantum memory—and (ii) the depth of the quantum circuit to be run before errors corrupt it. Since qubits are expensive, and coherence times and gate fidelity are limited, it is hugely important to save on both of these resources in order to develop a computing system that can solve practical problems in quantum chemistry.

In quantum chemistry applications of NISQ systems, such as for example VQE (the Variational Quantum Eigensolver), a quantum advantage is that quantum memory can store large quantum states with exponentially fewer memory resources than classical memory. This means that, in principle, NISQ devices can be used to study molecular systems that cannot be stored in classical memory.

VQE is a classical-quantum hybrid algorithm. Given a parametrized set of quantum states, VQE can use a classical optimizer to find the parameters of the minimum energy state. Knowledge of the ground state of a quantum system has many potential applications, such as in catalyst development and drug discovery.

Depending on the optimizer, the cost of this classical optimization can be exponential in the dimensionality of the space of parameters considered.

Since quantum states are characterized by an exponential number of classical parameters, they can be exponentially complex. Even if they can be stored efficiently, it takes a circuit of exponential depth to write a generic quantum state into quantum memory, which may make it impossible for a NISQ device.

In addition, it may be unfeasible to use VQE on such exponential sets states, because (i) the classical optimization runtime is too long on a parameter space of exponential size, and (ii) it is too costly to store the parameters in classical memory.

For VQE-like algorithms to provide quantum advantage, and hence become useful, it may be possible to work with sets of quantum states that are too costly to store in classical memory, but that are characterized by a small number of parameters. Such states are written into quantum memory with shallow quantum circuits, and VQE optimizes their energy classically with a reasonable runtime.

Discovering useful such sets of states is an active area of research. Unitary Coupled Cluster (UCC) is one of such sets of states in widespread use.

According to this disclosure, it is possible to construct an algorithm that utilizes any symmetries of a chemical, physical, or molecular system to reduce the dimensionality of the parameter space of the states to be written into quantum memory.

Having a smaller parameter space means that the complexity of the generic quantum state is lower, therefore given a fixed circuit depth for writing states into quantum memory, a larger class of states can be explored, compared to ignoring chemical symmetries. Given a set of states to be written into quantum memory, this algorithm can discard those states without appropriate symmetry properties. The states remaining depend on fewer parameters, and so can be written into quantum memory with shallower circuits, and hence in a shorter time and with a smaller error rate. The algorithm/method enables the determination of the structure of quantum circuitry required to write the states into quantum memory, that is, the types of quantum gates and how they can be connected to each other to form the circuit architecture required to interact with the quantum memory in a new and improved way.

Reducing the parameter space of the states under consideration gives a second advantage for VQE-like algorithms: in a smaller parameter space the classical optimizer needs fewer queries to quantum memory to find the minimum energy state, reducing the runtime.

The space of quantum states (Hilbert space) of symmetric molecules can be separated into different subspaces according to their symmetry properties. It is a theorem of quantum mechanics that, for the purposes of studying the energy of the molecule, the symmetry subspaces do not mix and hence can be considered independently.

A disclosed method takes any previously constructed set of states and reduces the dimensionality of this set by (i) discarding states without definite symmetry properties, and (i) grouping the remaining ones according to their symmetries.

These advantages are illustrated below for UCCSD (unitary coupled cluster singles-doubles)—a conventional set of states used in VQE. However, the present method works for any set of states.

The reduction of the number of parameters can be achieved through an automated algorithmic procedure from group theory known as ‘group averaging’ or ‘projecting into the symmetric representation’.

Effectively, this reduces the number of parameters describing the states to be explored to a fraction of the original set of parameters. The specific fraction depends on the specific symmetry of the molecule or physical system to be studied.

This means that, in some cases, it is possible to reduce the depth of the quantum circuits that write the states into quantum memory by a factor of 8, and the runtime of VQE by a power of ⅛—from t to t^{1/8}. It will be appreciated that the architecture of the quantum circuitry determined according to the present method can be very different to that which would be used according to conventional methods.

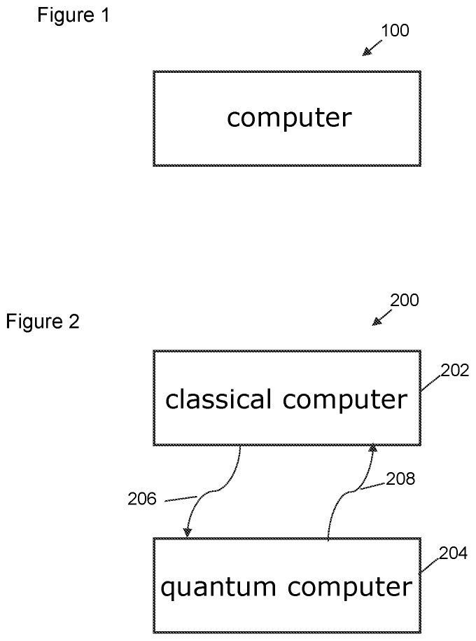

FIG. 1 shows a computer system 100 configured to determine a quantum circuit architecture according to the present disclosure. The quantum circuit architecture may be referred to as a compressed quantum circuit architecture because it can be shorter or be of reduced depth compared to conventional architectures. That is, the number of quantum logic gates may be reduced and the structure or topology of the connections between the gates may consequently also be different than those of conventional architectures.

The compressed quantum circuit architecture is determined so that it can be implemented in a quantum computer. However, the determination of the compressed quantum circuit architecture can be implemented on a classical computer.

Some advantages of the present disclosure are based on a point-symmetry group of a physical system that can be investigated using the quantum computer. The physical system can be a molecule, a combination of two or more molecules (such as a catalyst molecule and a reactant molecule) or could be the unit cell of a crystalline material, for example.

The computer system 100 is configured to receive the point-symmetry group from some outside source of information. The point symmetry group comprises a plurality of elements, each of which corresponds to a symmetry operation on the physical system and therefore also on all quantum basis states of the physical system. The point-symmetry group may include at least one non-trivial proper rotation. That is, there can be at least one rotation that maps the physical system into itself in addition to the trivial symmetry operation of rotating by 2π.

The computer system 100 is configured to receive a unitary operator, which is based on a plurality of parameters, where the unitary operator can operate on quantum basis states of the physical system. The unitary operator encodes a quantum circuit architecture designed to be applied to a quantum memory that can store the quantum basis states.

In the following discussion, the disclosed method is illustrated by considering Hamiltonian operators, but it will be appreciated that the disclosed method may also be advantageous in relation to other types of operators such as the density operator which relates to the density of electrons in a physical system.

In quantum mechanics, Hamiltonians H with symmetries are block-diagonal matrices acting on the space of quantum states (Hilbert space). It is possible to study their spectrum, e.g. the ground state, by studying each of the blocks independently.

The blocks of H correspond to irreducible representations (irreps) of the symmetry group G of the Hamiltonian H. States belonging to one block all transform with the same irrep r of G under symmetry transformations.

Given a reference state |ref [symbol] , it is possible to write the set of states of interest as U(θ)|ref [symbol] , where U(θ) is a unitary matrix depending on the plurality of parameters θ. This matrix encodes the quantum circuit that needs to be applied to a quantum memory unit containing the reference state |ref [symbol] to write a target state U(θ)|ref [symbol] .

Given a state |ref [symbol] in one block of H, it is possible to explore only that block with ansatz states of the type U(θ)|ref [symbol] by demanding that U(θ) is invariant under G. Invariance can be guaranteed by ‘group-averaging’ (also see eq. 25 and 26)) U(θ):

U(θ)→U̅(̅θ̅)̅=Σ_{g∈G}g(U(θ)). (1)

where g are the elements of G.

The present disclosure applies such savings to molecular symmetries. The group-averaging algorithm works for any symmetry group.

The computer system 100 can determine a symmetrized-unitary operator based on the unitary operator by using group averaging. The symmetrized-unitary operator therefore transforms as the identity representation of the point symmetry group. Since the physical system has some non-trivial point group symmetry, the symmetrized-unitary operator will be based on a proper subset only of the plurality of parameters, i.e. it will not depend on all of the plurality of parameters θ. Since the unitary operator encodes the quantum circuit, the group averaged symmetrized-unitary operator will encode a compressed quantum circuit architecture that has a reduced circuit depth compared to the quantum circuit. This reduction in circuit depth advantageously reduces the effects of decoherence and thereby enables a quantum computer to solve problems that would otherwise be physically impossible to solve because of decoherence effects.

In some examples, the unitary operator can be an exponential of an anti-Hermitian operator. In such cases the symmetrized-unitary operator can be determined by exponentiating a symmetrized-anti-Hermitian operator. The symmetrized-anti-Hermitian operator can be determined by averaging the anti-Hermitian operator over the plurality of elements of the point-symmetry group.

FIG. 2 shows a distributed computer system 200. The computer system 200 comprises a classical computer 202 operatively coupled to a quantum computer 204. The operative coupling means that the classical computer 202 can send information via a first link 206 to the quantum computer 204, while the quantum computer 204 can send information to the classical computer 202 via a second link 208. It will be appreciated that the first 206 and second 208 links may be solid connections or wireless connections.

Having determined the symmetrized-unitary operator, the classical computer 202 can then transmit the symmetrized-unitary operator to the quantum computer 204. This transmission of information, which may represent the symmetrized-unitary operator according to any form of encoding, can enable the quantum computer 204 to configure the compressed quantum circuit architecture by altering which quantum gates are connected to each other according to what topology. The quantum computer 204 can then apply the compressed quantum circuit architecture to a quantum memory containing a first quantum basis state of the physical system stored in a plurality of qubits. If the first quantum basis state transforms according to a first irreducible representation of the point-symmetry group, then the quantum computer 204 can investigate the properties of the block of the block diagonal unitary operator corresponding to the first irreducible representation of the point-symmetry group.

Symmetry-neutral ansatz operators are a subset of all ansatz operators U(θ) and hence they have fewer parameters. It is possible to express this fact mathematically as U̅(̅θ̅)̅=U (θ̅) for θ̅⊂θ: symmetry-preserving unitary matrices are a subset of all unitary matrices.

Having fewer parameters in the ansatz space saves quantum resources in quantum computers, as it takes fewer gates to write states of interest U(θ̅)|ref [symbol] into quantum memory, because the states of interest span a space of smaller dimension.

Having an ansatz space of smaller dimension U(θ̅)|ref [symbol] implies that, on top of states being prepared more efficiently, variational algorithms like VQE find the ground state faster, because they need to minimize the energy in a smaller space.

The quantum computer 204 can prepare a first quantum basis state (such as |ref [symbol] ) in the quantum memory and then receive the symmetrized-unitary operator U̅(̅θ̅)̅ from the classical computer 202. Thereby, the quantum computer 204 can prepare a compressed quantum circuit based on the symmetrized-unitary operator, by configuring connections between an appropriate selection of quantum logic gates. The compressed quantum circuit can then be applied to the quantum memory. This enables the quantum computer 204 to determine a first plurality of qubit measurement values for the first quantum basis state. It will be appreciated that before qubit measurement values can be determined, the quantum computer applies a second quantum circuit to the quantum memory, where the second quantum circuit encodes an operator relevant to an observable, such as the Hamiltonian where the observable is the energy.

Having obtained the first plurality of qubit measurement values, the quantum computer 204 can then transmit them, via any appropriate encoding scheme, to the classical computer 202.

The classical computer 202 may then estimate an expectation of the Hamiltonian operator acting on the first quantum basis state of the physical system based on the first plurality of qubit measurement values.

It is possible to change the block of the Hamiltonian Hᵣ that is explored by the ansatz U(θ̅)|ref [symbol] by acting on the reference state with U_{r′←r}—a unitary operator that changes the symmetry subspace of the reference state from r to the one of the irrep r′:

|ref′ [symbol] =U_{r′←r}|ref [symbol] (2)

The r′ block is explored similarly to the r block, with a symmetric U(θ̅) in the ansatz: U(θ̅)|ref′ [symbol] .

It will be appreciated that there are various ways the U_{r′←r} can be constructed.

Thus, the quantum computer 204 can prepare a second quantum basis state in the quantum memory, where the second quantum basis state transforms according to a second irreducible representation of the point-symmetry group that is different to the first irreducible representation. The quantum computer can apply the compressed quantum circuit to the quantum memory again and then determine a second plurality of qubit measurement values for the second quantum basis state. Once again, the quantum computer 204 can transmit the second plurality of qubit measurement values to the classical computer 202 via the second link 208.

The classical computer 202 can vary the plurality of parameters of the compressed unitary and then estimate an optimized-eigenvalue of the Hamiltonian by successively controlling the quantum computer to prepare varied compressed quantum circuits and apply each in turn of the varied compressed quantum circuits to the quantum memory containing each in turn of the quantum basis states of the physical system. Variation of the parameters can be undertaken by any appropriate method; the use of artificial intelligence methods, such as for example genetic algorithms, may be advantageous.

The computer system 200 may be used solve a wide variety of practical problems in the field of chemistry, such as, for example, catalyst development, drug discovery or materials development as discussed further below.

In an example, the physical system, to be studied using the computer system 200, may be a molecular system comprising one or more molecules. A molecule may be a catalyst, in which case the computer system 200 may determine a reduction in activation energy where the catalyst interacts with one or more reactant molecules. On an industrial scale, ammonia (NH₃) is produced using the Haber process, which operates at high temperatures and pressures and which therefore uses a great deal of energy. It has been estimated that ammonia production uses approximately 2% of global energy supply and accounts for approximately 3% of global carbon footprint. However, it is known that some plants, such as some legumes, provide a habitat for certain bacteria that convert atmospheric nitrogen and water into ammonia by a process that operates at standard temperature and pressure. Quantum computer-based simulations could be used to develop artificial organic catalysts that emulate this natural process on an industrial scale, thereby saving prodigious amounts of energy.

The computer system 200 could be used to simulate protein-molecule interactions for the purpose of developing new pharmaceutical medicaments. An advantage provided by using quantum simulation in this way is that vastly greater numbers of candidate molecule may be investigated than would be feasible by convention chemical methods.

In the field of materials development, the physical system may be the unit cell of a crystalline material. It will be appreciated that, if quantum basis states for the unit cell have matching boundary conditions on opposing surfaces of the unit cell then it may be possible to simulate bulk properties of new materials, which may lead to the design of materials with improved properties.

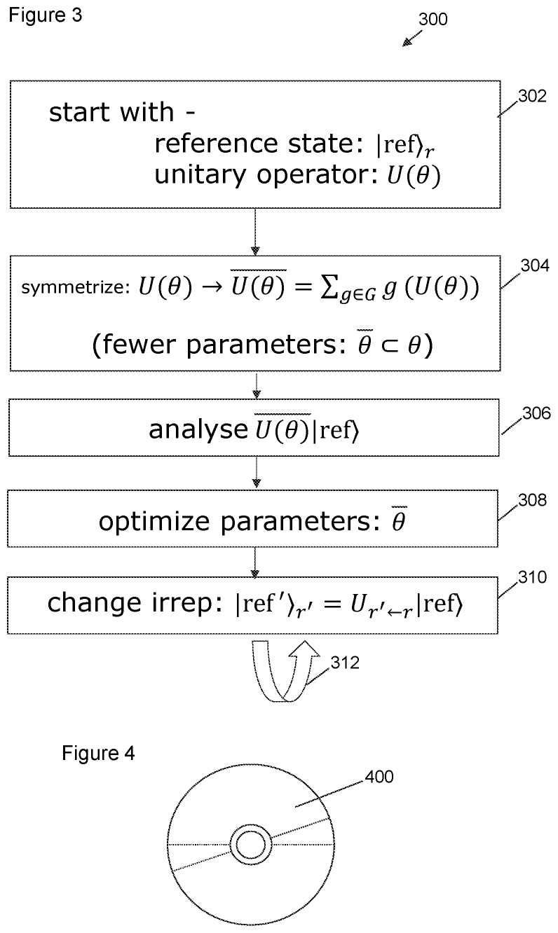

FIG. 3 shows a method 300 for determining a compressed quantum circuit architecture, for a quantum computer, based on a point-symmetry group of a physical system. The method 300 begins at a first step 302 with a quantum basis state re) (which transforms as a first irreducible representation of the point-symmetry group and therefore corresponds to a first block of the block diagonal Hamiltonian of the physical system) and a unitary operator U(θ) that depends on a plurality of parameters θ.

At a second step 304, the unitary operator U(θ) is group-averaged to determine a symmetrized-unitary operator U̅(̅θ̅)̅ that transforms as the identify representation of the point-symmetry group. It can be observed that the number of parameters of the symmetrized-unitary operator is a proper subset of the parameters of the unitary operators, i.e. the symmetrized-unitary operator does not depend on all of the parameters of the unitary operator. This will ensure that the compressed quantum circuit architecture that can be prepared in the quantum computer will be shorter and contain fewer quantum gates than would be possible if the circuit was prepared based on the unitary operator, since the unitary operator is based on a strictly greater number of parameters.

At a third step 306 the quantum basis state |ref [symbol] can be analyzed by applying a compressed quantum circuit that encodes the symmetrized-unitary operator U̅(̅θ̅)̅ to the quantum memory containing the quantum basis state |ref [symbol] . This can provide a plurality of qubit measurement values relating to the quantum basis state |ref [symbol] .

At a fourth step 308 the parameters θ̅ can be optimized by a classical computer. It will be appreciated that, at this point, the method can repeat the analysis step 306 (with a symmetrized-unitary operator based on the optimized parameters) and then the optimization step 308 as many times as required to achieve sufficient optimization of the parameters to provide satisfactory qubit measurement values for the quantum basis state |ref [symbol] to enable estimation of the required eigenvalues.

At step five 310 the first quantum basis state, and hence the relevant block of the Hamiltonian, is changed to a second quantum basis state that transforms as a second, different, irreducible representation of the point-symmetry group.

The sixth step 312 simply involves returning to the first step 302 but using the new second quantum basis state. In this way, it is possible to iterate through all of the irreps of the point-symmetry group, and thus all of the blocks of the Hamiltonian. This can generate the information needed to determine estimations for the expectations of the Hamiltonian for the physical system, as discussed above in relation to FIG. 2.

FIG. 4 shows an example computer program product 400 that contains instructions which, when executed, cause an apparatus, as described in FIG. 1, to at least perform steps of the method described above in relation to FIG. 3. Equivalently, there may also be provide a computer readable memory medium corresponding to the computer program product 400.

The following sections provide some specific examples and details of implementation of the present disclosure.

Quantum computational chemistry uses quantum computers to study electrons in molecules. Since the fundamental memory unit in a quantum computer is the qubit, the first step is a protocol to store electronic states in qubit memory.

States of N electrons can be described in ‘first quantisation’ as completely antisymmetric wavefunctions of their positions and spins, ψ(x₁, s₁; . . . ; x_{N}, s_{N}). Since the quantum computer is digital, while the description in terms of the wavefunction is continuous, it is necessary to implement a discretization to store the electronic state in qubit memory. One possibility is to discretize space with a lattice grid.

In ‘second quantization’, electronic states are described by occupation numbers of ‘molecular orbitals’. Molecular orbitals are one-electron states in the molecule, and there are infinitely many of them; but it is a good approximation to use only a finite number. To a set of M such orbitals it is possible to associate a set of anticommuting operators:

{bᵢ,bⱼ}=0, {bᵢ,bⱼ^{†}}=δᵢⱼ, i,j=1, . . . ,M, (3)

and consider the Fock space generated by the b^{†} operators acting on the Fock vacuum bᵢ|0. . . 0 [symbol] .

The quantum state of N electrons is then described as an excitation of the vacuum:

|ψ [symbol] =Σ_{{n}_{₁}_{, . . . ,n}_{_{N}}_{}}cₙ_{₁} _{. . . n}_{_{N}}bₙ_{₁}^{†} . . . bₙ_{_{N}}^{†}|0 . . . 0 [symbol] , (4)

where nᵢ=1, . . . , M belongs to the set of orbitals. The antisymmetry of the state (4) is automatically implemented by the anticommutation rules (3).

The Fock space of M orbitals is 2^{M}-dimensional (and the Hilbert space of N electrons, eq. (4), is

$$( \begin{matrix}M \\ N\end{matrix} ) - dimensional ) .$$

This non-polynomial scaling makes it unfeasible to store large electronic states classically. By contrast, in a quantum computer, such states can be stored in M qubits. For example, in the so-called Jordan-Wigner encoding, each qubit is associated with the occupation number of each orbital. It is possible to encode the electronic state bᵢ^{†}|0 . . . 0 [symbol] as the qubit state

|0 [symbol] ₁ [symbol] . . . [symbol] |0 [symbol] ᵢ₋₁ [symbol] |1 [symbol] ᵢ [symbol] |0 [symbol] ᵢ₊₁ [symbol] . . . [symbol] |0 [symbol] _{M}. (5)

Another possible encoding is the so-called ‘parity encoding’, in which the qubit j stores the sum of the occupation numbers (mod 2) of the i≤j orbitals. Hence, the state bᵢ^{†}|0 . . . 0 [symbol] reads

|0 [symbol] ₁ [symbol] . . . [symbol] |0 [symbol] ᵢ₋₁ [symbol] |1 [symbol] ᵢ [symbol] |1 [symbol] ᵢ₊₁ [symbol] . . . [symbol] |1 [symbol] _{M}. (6)

Other possible encodings are Bravyi-Kitaev and Bravyi-Kitaev tree, in which qubit j generically stores the parity of nearby orbitals i≲j.

Qubit encodings can also produce qubit Hamiltonians from substitution of bᵢ and bⱼ^{†} in the chemistry Hamiltonian by their representatives in the qubit Hilbert space. Generically:

H=Σₘ₌₁^{r}hₘσₘ, (7)

where σₘ are Pauli strings on the Hilbert space of M-qubits (that is, operators of the type σ₁^{a}^{₁} [symbol] . . . [symbol] σ_{M}^{a}^{_{M}}, with a₁, . . . , a_{M}=x, y, z), and there are r of them.

In NISQ implementations of quantum computational chemistry, it may be possible to store in qubit memory a quantum state defined by a polynomial (in M) number of parameters. The advantage of quantum computers is that they can efficiently store states that spread over non-classical regions of the Hilbert space. That is, with support over a large number of basis states. Given this large spread over Hilbert space, these states cannot be stored in classical memory. But, since they are defined by a small number of parameters, these parameters can be stored, and manipulated, classically.

There are several strategies for defining such electronic states. One of them is the so-called unitary-coupled cluster (UCC) ansatz:

|ψ [symbol] =e^{T-T}^{^{†}}|ref [symbol] , (8)

where |ref [symbol] is a ‘classical’ reference state (e.g., the Hartree-Fock state), and

T=Σcᵢⱼbᵢ^{†}bⱼ+cᵢⱼₖₗbᵢ^{†}bⱼ^{†}bₖbₗ+ . . . . (9)

It is possible to interpret the operator T−T^{†} as exciting orbitals near the reference state. A commonly used approximation truncates T to the order shown, in which case the ansatz (9) is called UCC singles-doubles (UCCSD). Notice that in this approximation the number of parameters needed to describe the state scales as (M−N)²·N² which is the number of cᵢⱼₖₗ parameters (as creation operators b^{†} must be in the unoccupied orbitals and annihilation operators b in the occupied ones in the reference state).

In Quantum Chemistry the spectrum of the electronic Hamiltonian operator H is of interest. For example, a basic question is what is the energy of the ground state of H?

Many interesting molecules have symmetries. These symmetry groups G are also symmetries of the Hamiltonian [G,H]=0. Therefore, eigenstates of H can be organised into irreps of G. In other words, the Hamiltonian is block diagonal, and each block is labelled by an irrep of G:

$$H = ( \begin{matrix}{H}_{{r}_{1}} & 0 & 0 & 0 \\ 0 & {H}_{{r}_{2}} & 0 & 0 \\ 0 & 0 & {H}_{{r}_{3}} & 0 \\ 0 & 0 & 0 & ⋱\end{matrix} ) . \qquad ( 10 )$$

In practice, this means that the problem of studying the spectrum of the Hamiltonian H can be broken down into the smaller problems of studying the spectrum of the Hamiltonians {Hᵣ_{ᵢ}} in the subspaces of the full Hilbert space spanned by each irrep of G.

Previous strategies for exploiting this fact in quantum computational chemistry fall broadly in two categories: (i) using chemical symmetries to modify the electronic Hamiltonian to penalize states outside a symmetry sector of the full Hilbert space; and (ii) using Abelian symmetries of the qubit Hamiltonian to save qubit memory resources by discarding qubits.

The disclosed method, instead, focusses on ansatz that explore each subspace rᵢ individually.

Below there is provided a brief discussion of relevant aspects of group theory.

An abstract group is a set of elements G={g₁, g₂, . . . } closed under a multiplication operation: g₁·g₂=g₃∈G.

This multiplication operation has an identity element e∈G and every element g∈G has an inverse g⁻¹∈G, g·g⁻¹=e.

A representation of a group can be a set of matrices {M₁, M₂, . . . } with the same matrix multiplication rules as the group elements: if Mᵢ represents gᵢ, then, eg, M₁·M₂=M₃, where ‘·’ here is ordinary matrix multiplication.

A representation is said to be irreducible if it does not preserve any subspace of the vector space on which its matrices act; otherwise it is reducible.

A standard problem in group theory is the Clebsch-Gordan problem, that asks what is the decomposition into irreducible representations (irreps) of the tensor product of irreps:

r₁ [symbol] r₂ [symbol] . . . =r₁′⊕r₂′⊕ . . . . (11)

As an example, consider SU(2), under which spin transforms. This group has infinitely many irreps, labelled by the eigenvalue of the total spin, S²=s(s+1). These irreps have dimension 2s+1. It is well-know that two spin ½ irreps—e.g., two electrons—can compose into a trivial singlet s=0 irrep, and a triplet s=1 irrep. Calling these irreps by their dimensionality (ie: s=0 by 1; s=½ by 2; and s=1 by 3), this fact can be expressed:

2 [symbol] 2=1⊕3. (12)

The symmetries of molecules are so-called point groups. These are subgroups of the Euclidean group that leave one point fixed. The group of symmetries of Euclidean space, which are translations and rotations, [symbol] O(3). These groups are well studied and their properties are tabulated. One example is c₃ᵥ, the group of symmetries of NH₃. These are the symmetries of the equilateral triangle in 2 dimensions. This group has six elements: identity, rotations by ±2π/3, and three reflections. As an abstract group, it is the group of permutations of three elements. It has three irreps: the totally symmetric one A₁, the alternating one A₂, and the standard one E; the first two are 1-dimensional, and the latter is 2-dimensional. They compose in a simple way, e.g.:

$${A}_{1}^{{m}_{1}} ⊗ {A}_{2}^{{m}_{2}} = \{ \begin{matrix}{A}_{1} & {m}_{2} even \\ {A}_{2} & {m}_{2} odd\end{matrix} . \qquad ( 13 )$$

In methods relating to symmetry modified electronic Hamiltonians it is possible to modify the Hamiltonian to focus on a particular symmetry sector. One way is by adding a penalty term:

H→H+μ(G−gᵣ_{ᵢ})², (14)

where μ>0 is a parameter whose role is to give extra energy to states that do not fall into the irrep of interest. G−gᵣ_{ᵢ} is schematic notation for an operator that has eigenvalue zero on the states that belong to the irrep rᵢ of interest, and different from zero otherwise.

There are variations of this method depending on the penalty function chosen.

Another possibility is to constrain to the subspace of the irrep of interest by projecting the Hamiltonian to that subspace

H→HPᵣ_{ᵢ}, (15)

where Pᵣ_{ᵢ} is a projector.

Non-relativistic molecular Hamiltonians conserve the number of electrons and the total spin of the electronic wavefunction, and therefore have at least these two symmetries. These can be used to save two qubits when storing the state in qubit memory: one for each symmetry.

This can be illustrated with the parity encoding method of eq. 6. Consider a system with

$$\frac{M}{2}$$

spatial orbitals—which become M electronic orbitals upon including the spin degree of freedom. Ordering the orbitals such that the first

$$\frac{M}{2}$$

are all spin up, and the second

$$\frac{M}{2}$$

are all spin down, we get that the

$$\frac{M}{2} t h$$

qubit is a measure of the total electronic spin (mod 2), [the total spin S_{z} is given by a combination of n_{↑} and the total number of electrons n_{↑}+n_{↓}, as 2n_{↑}−(n_{↑}+n_{↓})=n_{↑}−n_{↑}.] and the last qubit counts the number of electrons:

$$\vert {n}_{1 ↑} 〉 ⊗ … ⊗ \vert {\sum }_{i = 1}^{\frac{M}{2} - 1} {n}_{i ↑} 〉 ⊗ \vert {n}_{↑} 〉 ⊗ \vert {n}_{↑} + {n}_{1 ↓} 〉 ⊗ … ⊗ \vert {n}_{↑} + {n}_{↓} 〉 , \\ {n}_{↑} ≡ {\sum }_{i = 1}^{\frac{M}{2}} {n}_{i ↑} , {n}_{↓} ≡ {\sum }_{i = 1}^{\frac{M}{2}} {n}_{i ↓} . \qquad ( 16 )$$

The fact that n_{↑} and n_{↑}+n_{↓} are conserved implies that the qubit Hamiltonian does not change their values, and therefore they can be ignored in qubit memory when studying the spectrum; the state can be stored in M−2 qubits. More specifically, the qubit Hamiltonian can only act on the

$$\frac{M}{2} t h$$

and Mth qubit of (6) via the σ^{z} Pauli operator, and therefore we can dispense with these qubits and substitute

$${σ}_{\frac{M}{2}}^{z}$$

and σ_{M}^{z} by their eigenvalues in the qubit Hamiltonian (7).

Similar savings can be made with Bravyi-Kitaev encodings. Note, however, that such savings are not so straightforward in the Jordan-Wigner encoding of eq. 5, as there are no qubits devoted to the conserved numbers n_{↑} and n_{↓}, or their combinations.

Below provides a disclosure relating to savings in ansatz space based on particle number and spin.

An objective is to explore the Hilbert spaces in the diagonal blocks {Hᵣ_{ᵢ}} of H of eq. 10 individually, as opposed to exploring all the Hilbert of H at once. Since the spaces in the block-diagonals are smaller, fewer parameters are required in the ansatz needed to explore them.

Having a smaller ansatz space is of crucial benefit in algorithms like VQE; it means that it takes faster for the classical optimizer to find the minima; it needs fewer queries to the quantum computer; plus these queries require shorter quantum circuits, as the ansatz has fewer terms.

In this section ansatz savings due to spin and particle number symmetries are presented.

Demanding that the ansatz of (8) only explores the subspace of the irrep of the reference state |ref [symbol] is equivalent to demanding that the T operator is neutral under the symmetry group. To see this, consider the action of the symmetry G on the UCC state, which is by a (generically reducible) unitary representation U_{G}:

U_{G}e^{T-T}^{^{†}}|ref [symbol] =U_{G}e^{T-T}^{^{†}}U_{G}^{†}U_{G}|ref [symbol] . (17)

The state |ref [symbol] can be considered to live in one of the blocks of H, and so it transforms to U_{G}|ref [symbol] by an irrep of G, say rᵢ. Generically, e^{T-T}^{^{†}} transforms to U_{G}e^{T-T}^{^{†}}U_{G}^{†} by a reducible representation of G. The UCC state will be in the same irrep as |ref [symbol] if e^{T-T}^{^{†}} is in the trivial (or totally symmetric) representation of G, denoted A₁ [A₁ is standard terminology for point-symmetry groups, by contrast, for continuous groups, as spin (SU(2)), the trivial representation is more commonly denoted 1] as A₁ composes trivially with any other irrep

A₁ [symbol] rᵢ=rᵢ. (18)

Equivalently:

[U_{G},e^{T-T}^{^{†}}]=0,⇒[U_{G},T]=0, (19)

so T should be neutral (ie, transform trivially) under the action of G.

The UCCSD ansatz, eq. 9, contains 1- and 2-particle excitations around a reference state (usually the Hartree-Fock state). 1-particle excitations are created with the operators {cᵢⱼbᵢ^{†}bⱼ}; 2-particle excitations are created with {cᵢⱼₖₗ bᵢ^{†}bⱼ^{†}bₖbₗ}. Notice that these operators conserve the number of electrons, because they contain as many annihilations as creation operators. In other words, the operators are in the trivial representation of number: they have number zero, [\hat{N},T]=0. For N electrons and M>>N orbitals, [this is the precision regime−large number of orbitals] the number of parameters in this ansatz is essentially the number of cᵢⱼₖₗs, which is ˜M²N². [The annihilation operators are the ones filled in the Hartree-Fock state, that is, the first N states. The creation operators are empty in Hartree-Fock, and there are M−N˜M of them.]

This already implements significant savings compared to generic 2- and 4-site operators, that would also contain particle number non-conserving terms like bᵢbⱼ and bᵢ^{†}bₖ^{†}bⱼ^{†}bₗ^{†}. In this case we would have ˜M⁴ parameters in the ansatz.

Further imposing conservation of spin requires that all terms in the ansatz have zero net spin. This modifies the particle-conserving counting above to ˜⅛M²N² parameters. To see this explicitly, notice that each term in the operators {cᵢⱼₖₗbᵢ^{†}bⱼ^{†}bₖbₗ} can be either spin up or down. That is 2⁴ spin options. Only 6 of these have net S_{z}=0: b_{i↑}^{†}b_{j↑}^{†}b_{k↑}b_{l↑}, b_{i↓}^{†}b_{j↓}^{†}b_{k↓}b_{l↓}, b_{i↑}^{†}b_{j↓}^{†}b_{k↑}b_{l↓}, b_{i↑}^{†}b_{j↓}^{†}b_{k↓}b_{l↑}, b_{i↓}^{†}b_{j↑}^{†}b_{k↑}b_{l↓}, b_{i↓}^{†}b_{j↑}^{†}b_{k↓}b_{l↑} (where we have separated the spin-orbital indices into their spatial and spin part); and only two linearly independent combinations have s=0:

$$\begin{matrix}{b}_{i ↑}^{†} {b}_{j ↑}^{†} {b}_{k ↑} {b}_{l ↑} + {b}_{i ↓}^{†} {b}_{j ↓}^{†} {b}_{k ↓} {b}_{l ↓} + \frac{1}{2} ( {b}_{i ↑}^{†} {b}_{j ↓}^{†} + {b}_{i ↓}^{†} {b}_{j ↑}^{†} ) ( {b}_{k ↓} {b}_{l ↑} + {b}_{k ↑} {b}_{l ↓} ) , & ( 20 ) \\ \frac{1}{2} ( {b}_{i ↑}^{†} {b}_{j ↓}^{†} - {b}_{i ↓}^{†} {b}_{j ↑}^{†} ) ( {b}_{k ↓} {b}_{l ↑} - {b}_{k ↑} {b}_{l ↓} ) . & ( 21 )\end{matrix}$$

Of the four other linearly independent S_{z}=0 combinations, one belongs in a spin 2 irrep and three have spin 1.

Therefore, out of the 2⁴ spin operators, only 2, ⅛th, have s=0 and thus are neutral under rotations.

Below, a deduction that (20) and (21) have s=0 is presented.

One possible way is noticing that (20) is

Σₛ_{_{z}}_{{-1,0,1}}|s=1,S_{z} [symbol] ᵢⱼ [symbol] s=1,S_{z}|ₖₗ, (22)

and the operator in (21) is

|s=0 [symbol] ᵢⱼ [symbol] s=0|ₖₗ. (23)

Since both of these operators are explicitly an average over all rotations, they are rotation invariant and hence have s=0 (for an expanded analysis of this see around eq. (26) below). Notice that, despite being projectors, both (20) and (21) are generically non-trivial in a subspace Hᵣ_{ᵢ} of total s fixed, as generically having total s fixed does not imply that any pair of spatial orbitals are in an eigenstate of their total spin—(Sₖₗ)² in this case.

Possible savings in ansatz space based on point-group symmetries are presented below.

In relation to eq. 19 it is argued that the T operator should be symmetry neutral. Assuming the use of SALCs (Symmetry-Adapted Linear Combination of orbitals), each of the creation and annihilation operators will belong in some irrep of the symmetry group of the molecule. [SALCs are molecular orbitals that have definite symmetry properties, for example they may transform in definite irreps under symmetry transformations. It is not necessary to use SALCs as non-symmetry adapted orbitals can also be used, but it can be advantageous to use SALC.] For the T operator to be in the trivial irrep, it can only be composed of terms bᵢ^{†}bⱼ^{†}bₖ^{†}bₗ whose joint transformation contains the trivial irrep:

Γᵢ [symbol] Γⱼ [symbol] Γ̅ₖ [symbol] Γ̅ₗ=A₁⊕ . . . . (24)

T is then made of the projection of (24) into the A₁ irreps in the right-hand side.

This can be ensured with a number of strategies. One is to use the representation theory of the relevant symmetry group to consider only sets of four irreps such that condition (24) is satisfied, and project them into A₁.

To illustrate these considerations, consider the symmetry of rotations of spin, SU(2). It is well known that 2 [symbol] 2 [symbol] 2 [symbol] 2=1⊕1⊕ . . . (where 2 denotes the spin ½, irrep; and 1 the trivial, or maximally symmetric, irrep: S²=0 [1 in SU(2) stands for the same concept as A₁ for point-symmetry groups]), and so we expect two linearly independent combinations of bᵢ^{†}bⱼ^{†}bₖbₗ to conserve spin—as indeed (20) and (21) do. On the other hand, these considerations rule out, e.g., products of an odd number of spin ½ operators, as these will transform in half-integer spin irreps—and they are in any case ruled out already by particle number conservation.

One can project to the A₁ irrep by ‘group averaging’. Given an operator O, we can construct a symmetry-neutral version of it by adding together the result of all its symmetry transformations:

O→Ō=Σ_{g∈G}g(O). (25)

The resulting operator Ō clearly is neutral under all symmetry transformations g∈G:

g(Ō)=Ō. (26)

This operation can be carried out regardless of whether A₁ appears in the right-hand side of (24); if it does not, e.g. if O is in an irrep other than A₁, Ō is zero. In practice, this means that it is possible to skip the step of selecting irreps that combine into A₁, as in (24), and directly group average a set of operators to obtain a subset of symmetry-neutral operators.

As an illustration consider spin again. Clearly, operators (20) and (21) can be seen as their average over the group, as all terms in each combination are generated from rotating one of them, e.g.:

Σ_{g∈SU(2)}g(|S=1,S_{z}=−1 [symbol] ᵢⱼ [symbol] s=1,S_{z}=−1|ₖₗ)=Σₛ_{_{z}}_{={-1,0,1}}|s=1,S_{z} [symbol] ᵢⱼ [symbol] s=1,S_{z}|ₖₗ. (27)

Note that the general method for constructing symmetry neutral Ts, eq. (26), explains savings made with spin and particle-number symmetries. The novelty of the present method is to apply these methods systematically to point-symmetry groups for wavefunction ansatz in quantum algorithms such as VQE.

Below are some examples relating to specific symmetry groups.

C₃ᵥ Symmetry: H₃⁺

C₃ᵥ, the group of symmetries of the equilateral triangle, is the simplest non-Abelian group.

This group has three irreps: The totally symmetric one, A₁, the alternating one, A₂, and the standard one, E. The latter has dimension 2, the other two have dimension 1.

Consider H₃⁺ in the equilateral triangle geometry—that is, two electrons orbiting three protons at the vertices of an equilateral triangle. Take just one spatial s orbital for each of the atoms: s₁, s₂, and s₃. These make three symmetry-adapted molecular orbitals, one of which is in A₁:

$${ϕ}_{{A}_{1}} = \frac{1}{\sqrt{3}} ( {s}_{1} + {s}_{2} + {s}_{3} ) , \qquad ( 28 )$$

and two of which make an E doublet:

$${ϕ}_{{E}_{1}} = \frac{1}{\sqrt{6}} ( 2 {s}_{3} - {s}_{1} - {s}_{2} ) , {ϕ}_{{E}_{2}} = \frac{1}{\sqrt{3}} ( {s}_{2} - {s}_{1} ) . \qquad ( 29 )$$

There are then a total of six spin-orbitals: ϕ_{A}_{₁}_{↑}, ϕ_{A}_{₁}_{↓}, ϕ_{E}_{₁}_{↑}, ϕ_{E}_{₁}_{↓}, ϕ_{E}_{₂}_{↑}, and ϕ_{E}_{₂}_{↓}.

C₃ᵥ has six elements: identity, rotations by 2π/3, rotations by 4π/3, and reflections along each median: P₁₂, P₁₃, P₂₃. The action of these elements is trivial on A₁, as A₁ is the totally symmetric irrep, and their action on

$$ψ = ( \begin{matrix}{ψ}_{{E}_{1}} \\ {ψ}_{{E}_{2}}\end{matrix} ) ∈ E$$

can be compactly expressed by considering the complex combination:

ψ_{c}≡ψ_{E}_{₁}+iψ_{E}_{₂}. (30)

Then:

(31)

| | 1 | $R ( \frac{2 π}{3} )$ | $R ( \frac{4 π}{3} )$ | P₁₂ | P₁₃ | P₂₃ |
|---|---|---|---|---|---|---|
| [symbol] | [symbol] | e^{i 2π/3} [symbol] | e^{i 4π/3} [symbol] | ( [symbol] )* | e^{-i 2π/3}( [symbol] )* | e^{-i 4π/3}( [symbol] )* |

Consider now the S²=0 “singles” operators b_{i↑}^{†}b_{j↑}+b_{i↓}^{†}b_{j↓}. We have argued above that we only need to consider combinations of orbital indices transforming in the A₁ irrep. From the representation theory of C₃ᵥ, we know that we can obtain an A₁ from either composing two A₁s:

A₁ [symbol] A₁=A₁ (32)

or from composing two Es:

E [symbol] E=A₁⊕A₂⊕E. (33)

Another possibility in C₃ᵥ would be A₂ [symbol] A₂=A₁, but this is irrelevant for the H₃⁺ example as it does not have any molecular orbitals in A₂.

The complex notation is especially useful in seeing how this representation theory works. Given two doublets ψ∈E, ψ′∈E, the A₁ representation of ψ [symbol] ψ′ is the combination:

[symbol]

( [symbol] )*+( [symbol] )* [symbol] =ψ₁ψ₁′+ψ₂ψ₂′. (34)

This is clearly in the A₁ irrep, as it is invariant under all operations in (31). Alternatively, notice that it is a scalar in space, and hence it is invariant under all C₃ᵥ operations which, like all point symmetry groups, form a subgroup of the Euclidean group in three dimensions.

The A₂ representation is given by

[symbol]

( [symbol] )*−( [symbol] )* [symbol] , (35)

and E is:

\sqrt{ψ}. (36)

It is straightforward to check using the rules (31) that irreps in A₂ and E group-average to zero. E.g.:

Σ_{g∈C}_{₃ᵥ}g( [symbol] )= [symbol] +e^{i2π/3} [symbol] +e^{i4π/3} [symbol] +( [symbol] )*+e^{-2π/3}( [symbol] )*+e^{-i4π/3}( [symbol] )=0. (37)

This can be seen as a check that the argument above, that group averaging projects to the A₁ irrep, is correct.

Therefore, there are in principle only two possible “singles” operators in S²=0 and A₁:

b_{A}_{₁}_{↑}^{†}b_{A}_{₁}_{↑}+b_{A}_{₁}_{↓}^{†}b_{A}_{₁}_{↓} (38)

which is the number (occupation) operator of the A₁ orbital, and

[symbol]

+ [symbol] =b_{E}_{₁}_{↑}^{†}b_{E}_{₁}_{↑}+b_{E}_{₂}_{↑}^{†}b_{E}_{₂}_{↑}+b_{E}_{₁}_{↓}^{†}b_{E}_{₁}_{↓}+b_{E}_{₂}_{↓}^{†}b_{E}_{₂}_{↓}, (39)

which also turns out to be a combination of number operators. Normally it is not necessary to keep operators in UCC that are combinations of number operators, so it is possible to discard (38) and (39).

For the “doubles” operators bᵢ^{†}bⱼ^{†}bₖbₗ, there exist the following totally symmetric combinations:

A₁ [symbol] A₁ [symbol] A₁ [symbol] A₁=A₁, (40)

which is actually zero or a set of number operators. We also have

E [symbol] E [symbol] A₁ [symbol] A₁=A₁⊕A₂⊕E, (41)

and similarly for A₁ [symbol] E [symbol] E [symbol] A₁ and A₁ [symbol] A₁ [symbol] E [symbol] E. (The distinction between these orderings is whether the operators in E and A₁ are in creation or annihilation slots.) Each of these combinations have a component in A₁ that is potentially not just a combination of occupation numbers.

Finally, for four Es

E [symbol] E [symbol] E [symbol] E=(A₁⊕A₂⊕E) [symbol] (A₁⊕A₂⊕E)=A₁^{⊕3}⊕A₂^{⊕3}⊕E^{⊕5}. (42)

The symmetry savings are as follows: Out of all the potential operators that can be constructed by picking four orbitals amongst E and A₁ irreps, of which there are 3⁴ options: the A₁ [symbol] ⁴ is in A₁; only 3 out of 2⁴ E [symbol] ⁴ operators are in A₁; only 6 out of 6·2² E [symbol] ² [symbol] A₁ [symbol] ² are in A₁; and none of 4·(2+2³) operators in E [symbol] A₁ [symbol] ³ and A₁ [symbol] E [symbol] ³ are totally symmetric. In summary, symmetry considerations keep only 10/81≈⅛ of the possible operators, before implementing any savings due to spin.

Combining with the savings from spin-symmetry, there are just ⅛²= 1/64 of all the 4-site operators.

Consider the Hartree-Fock state of H₃⁺ is b_{A}_{₁}_{↑}^{†}b_{A}_{₁}_{↓}^{†}|0 [symbol] , and consider only combinations of operators that just have A₁ annihilation operators. Furthermore, since the HF state has spin zero, only the spin indices in (21) are relevant. Discarding the operators that are just combinations of occupation numbers, there is just one UCCSD operator to consider, the A₁ in E [symbol] E [symbol] A₁ [symbol] A₁:

T=(b_{E}_{₁}_{↑}^{†}b_{E}_{₁}_{↓}^{†}+b_{E}_{₂}_{↑}^{†}b_{E}_{₂}_{↓}^{†})b_{A}_{₁}_{↑}b_{A}_{₁}_{↓}. (43)

The UCC ansatz indeed suffices to explore the subspace of H₃⁺ of zero spin and A₁ symmetry, which is two dimensional:

e^{α(T-T}^{^{†}}⁾b_{A}_{₁}_{↑}^{†}b_{A}_{₁}_{↓}^{†}|0 [symbol] =(cos αb_{A}_{₁}_{↑}^{†}b_{A}_{₁}_{↓}^{†}+sin α(b_{E}_{₁}_{↑}^{†}b_{E}_{₁}_{↓}^{†}+b_{E}_{₂}_{↑}^{†}b_{E}_{₂}_{↓}^{†}))|0 [symbol] . (44)

In this case, symmetry arguments—molecular and spin—go from naively 4·2²=16 “doubles” parameters if symmetry is ignored (that is, the doubles that do not annihilate b_{A}_{₁}_{↑}^{†}b_{A}_{₁}_{↓}^{†}|0 [symbol] and that do not contain number operators) to just 1 parameter. Disregarding molecular symmetry, there would be 3 operators after having considered spin savings and absence of number operators—in this case molecular symmetries this divides the number of generators by an extra factor of 3.

[symbol]

₂ Symmetries; H₄

Consider the case of ( [symbol] ₂)ⁿ molecular symmetries. These symmetries are reflections. We demand that all terms in the VQE ansatz are neutral under the reflections, i.e., even. For each [symbol] ₂ this discards half of the otherwise possible terms—the odd ones—dividing by two the number of operators in the ansatz [the parity of the {cᵢⱼₖₗ bᵢ^{†}bⱼ^{†b}ₖbₗ} operators is the product of the parities of all operators, the odd operators are the ones containing only one odd b or b^{†}, or only one even b or b^{†}, which is a total of 8 cases: half of the 2⁴ possibilities]. Therefore, for a ( [symbol] ₂)ⁿ molecular symmetry, present arguments discard ½ⁿ operators that would otherwise be required. Adding this to the spin savings, only ½³⁺ⁿ of the operators.

It is possible to illustrate these considerations with an example. Take H₄ in a parallelogram geometry. Its symmetry group is ( [symbol] ₂)², the reflections across each of the diagonals. The four symmetry-adapted orbitals coming from the s orbitals around each H are:

ϕₑₑ=s₁+s₃,

ϕₒₑ=s₁−s₃,

ϕ_{ee′}=s₂+s₄,

ϕₑₒ=s₂−s₄. (45)

The labels specify whether the orbitals are even or odd under each of the [symbol] ₂ symmetries (P₁₃ and P₂₄ resp).

Symmetry-neutral operators are even under all reflections. In UCC, there are the following reflection-neutral singles combinations: (ee,ee), (ee′,ee′), (ee,ee′), (ee′,ee), (eo,eo), (oe,oe). Of these, the only ones that at spin-0 are not number operators are (ee,ee′), (ee′,ee):

b_{ee↑}^{†}b_{ee′↑}+b_{ee↓}^{†}b_{ee′↓},

b_{ee′↑}^{†}b_{ee↑}+b_{ee′↓}^{†}b_{ee↓}. (46)

However, assuming the Hartree-Fock state is b_{ee↑}^{†}b_{ee↓}^{†}b_{eo↑}^{†}b_{eo↓}^{†}|0 [symbol] , none of the operators in (46) is non-trivial.

The neutral “doubles” operators need to have an even number of each of the odd operators eo or oe resp. Out of the 4⁴ ijkl possibilities: 2⁴ are even because they have no eos or oes;

$$( \begin{matrix}4 \\ 2\end{matrix} ) \cdot {2}^{2}$$

have 2 eos and no oes;

$$( \begin{matrix}4 \\ 2\end{matrix} ) \cdot {2}^{2}$$

have 2 oes and no eos;

$$( \begin{matrix}4 \\ 2\end{matrix} )$$

have oes and 2 eos; 1 has 4 eos; and 1 has 4 oes. That is, a total of 72/4⁴≈¼ of all the operators are neutral. This is expected given that ( [symbol] ₂)² has 4 elements; 1, P₁₃, P₂₄, P₁₃P₂₄. Making the further simplifications of discarding number operators, and implementing the savings coming from spin symmetry, there exist only 6 non-trivial reflection-even spin-0 operators acting on the assumed HF state:

(b_{ee′↑}^{†}b_{ee′↓}^{†}−b_{ee′↓}^{†}b_{ee′↑}^{†})(b_{ee↑}b_{ee↓}−b_{ee↓}b_{ee↑})

(b_{ee′↑}^{†}b_{ee′↓}^{†}−b_{ee′↓}^{†}b_{ee′↑}^{†})(b_{eo↑}b_{eo↓}−b_{eo↓}b_{eo↑})

(b_{eo′↑}^{†}b_{eo′↓}^{†}−b_{eo′↓}^{†}b_{eo′↑}^{†})(b_{ee↑}b_{ee↓}−b_{ee↓}b_{ee↑})

(b_{eo′↑}^{†}b_{eo′↓}^{†}−b_{eo′↓}^{†}b_{eo′↑}^{†})(b_{eo↑}b_{eo↓}−b_{eo↓}b_{eo↑}). (47)

The Hilbert space in the irrep of the HF state is spin-0 and reflection-neutral, and it has dimension 8, so in this case the UCCSD ansatz just does not explore all the spin-0 and reflection-neutral subspace of the HF state. This is of course what happens in general—truncated UCC ansatz only cover a portion of the Hilbert space.

In this case, symmetry considerations truncate the number of non-trivial doubles on b_{ee↑}^{†}b_{ee↓}^{†}b_{eo↑}^{†}b_{eo↓}^{†}|0 [symbol] from

$${( \begin{matrix}4 \\ 2\end{matrix} )}^{2} = 36$$

down to 4. Ignoring molecular symmetry, there would be 6 operators, so in this case molecular symmetry gives an extra saving factor of 1.5.

So far the state e^{T-T}^{^{†}}|ref [symbol] has been in the same irrep as |ref [symbol] . To explore other irreps, it is necessary to first act on |ref [symbol] with an operator U_{r′←r} that puts it in another irrep

|ref′ [symbol] =U_{r′←r}|ref [symbol] (48)

and then act with a symmetry-neutral e^{T-T}^{^{†}} operator on |ref′) to explore the subspace of the irrep of |ref′ [symbol] .

Another important observation is that, for non-Abelian symmetry groups, the Hartree-Fock state is not expected to be in an irrep of the symmetries. That is because non-Abelian symmetry groups have irreps of dimensionality>1, which makes the Clebsch-Gordan problem non-trivial. That is, a HF state that fills orbitals in irreps |r₁, r₂, . . . , r_{N} [symbol] generically will be in a mixture of irreps:

r₁ [symbol] r₂ [symbol] . . . [symbol] r_{N}=r₁′⊕r₂′⊕ . . . . (49)

To explore a single irrep rᵢ′ requires projecting the Hartree-Fock state to that irrep.

This comment is not relevant for spin symmetry, as the closed-shell Hartree-Fock state always has S²=0 (and open shell spin ½). It also trivialises for Abelian groups, as for these all irreps have dimension=1 and hence the right-hand side of the Clebsch-Gordan equation above only has one term, which means that the HF state is always in a single irrep of the symmetry group:

r₁ [symbol] r₂ [symbol] . . . [symbol] r_{N}=r₁′. (50)

The present disclosure provides a new strategy for saving resources in quantum computational chemistry by determining novel quantum circuit architectures that are shorter/shallower than conventional alternatives. The strategy can exploit the symmetry of molecules to find the spectrum of their Hamiltonian with VQE with fewer and shorter queries to a quantum computer required. The strategy can be advantageously applied to all non-trivial point group symmetries.

1. A computing system configured to determine a compressed quantum circuit architecture, for a quantum computer, based on a point-symmetry group of a physical system, the computing system comprising a classical computer operatively coupled to the quantum computer, wherein the classical computer is configured to:
receive the point-symmetry group, wherein the point-symmetry group comprises a plurality of elements, each element corresponding to a symmetry operation on all quantum basis states of the physical system;
receive a unitary operator based on a plurality of parameters, wherein the unitary operator encodes a quantum circuit architecture;
determine a symmetrized-unitary operator based on the unitary operator, wherein the symmetrized-unitary operator:
transforms as the identity representation of the point-symmetry group;
is based on a proper subset only of the plurality of parameters; and
encodes the compressed quantum circuit architecture; and
transmit the symmetrized-unitary operator to the quantum computer to enable configuration of the compressed quantum circuit architecture and application of the compressed quantum circuit architecture to a quantum memory containing a first quantum basis state of the physical system stored in a plurality of qubits, wherein the first quantum basis state transforms according to a first irreducible representation of the point-symmetry group.

2. The computing system of claim 1 further comprising the quantum computer, wherein the quantum computer is configured to:
prepare the first quantum basis state in the quantum memory;
receive the symmetrized-unitary operator;
prepare a compressed quantum circuit based on the symmetrized-unitary operator;
apply the compressed quantum circuit to the quantum memory;
determine a first plurality of qubit measurement values for the first quantum basis state; and
transmit the first plurality of qubit measurement values to the classical computer.

3. The computing system of claim 2, wherein the quantum computer is further configured to:
prepare a second quantum basis state in the quantum memory, wherein the second quantum basis state transforms according to a second irreducible representation of the point-symmetry group different to the first irreducible representation;
apply the compressed quantum circuit to the quantum memory;
determine a second plurality of qubit measurement values for the second quantum basis state; and
transmit the second plurality of qubit measurement values to the classical computer.

4. The computing system of claim 2, wherein the classical computer is configured to estimate an expectation of a quantum mechanical operator, for the physical system, based on the first plurality of qubit measurement values.

5. The computing system of claim 4, wherein the classical computer is configured to vary one or more of the plurality of parameters and estimate an optimized-eigenvalue of the quantum mechanical operator by successively controlling the quantum computer to prepare one or more varied compressed quantum circuits and apply each in turn of the one or more varied compressed quantum circuits to the quantum memory containing each in turn of one or more of the quantum basis states of the physical system.

6. The computing system of claim 4, wherein the quantum mechanical operator is a Hamiltonian operator.

7. The computing system of claim 1 wherein the symmetrized-unitary operator is determined by averaging the unitary operator over the plurality of elements of the point-symmetry group.

8. The computing system of claim 1, wherein the unitary operator is an exponential of an anti-Hermitian operator, and the symmetrized-unitary operator is determined by exponentiating a symmetrized-anti-Hermitian operator determined by averaging the anti-Hermitian operator over the plurality of elements of the point-symmetry group.

9. The computing system of claim 1, further configured to determine a reduction in activation energy in catalyst development, wherein:
the physical system is a molecular system;
the classical computer is configured to vary one or more of the plurality of parameters and estimate an optimized-eigenvalue of a Hamiltonian operator of the molecular system by successively controlling the quantum computer to prepare one or more varied compressed quantum circuits and apply each in turn of the one or more varied compressed quantum circuits to the quantum memory containing each in turn of one or more of the quantum basis states of the physical system.

10. The computing system of claim 1, further configured to simulate protein-molecule interaction, wherein:
the physical system is a molecular system; and
the classical computer is configured to vary one or more of the plurality of parameters and estimate an optimized-eigenvalue of a Hamiltonian operator of the molecular system by successively controlling the quantum computer to prepare one or more varied compressed quantum circuits and apply each in turn of the one or more varied compressed quantum circuits to the quantum memory containing each in turn of one or more of the quantum basis states of the physical system.

11. The computing system of claim 1, further configured to perform materials development, wherein:
the physical system is a unit cell of a crystalline material; and
the classical computer is configured to vary one or more of the plurality of parameters and estimate an optimized-eigenvalue of a Hamiltonian operator of the physical system by successively controlling the quantum computer to prepare one or more varied compressed quantum circuits and apply each in turn of the one or more varied compressed quantum circuits to the quantum memory containing each in turn of one or more of the quantum basis states of the physical system.

12. The computing system of claim 1, further configured to perform any one or more of catalyst development, drug discovery or materials development.

13. The computing system of claim 1, wherein the point-symmetry group comprises at least one non-trivial proper rotation.

14. A computer-implemented method for determining a compressed quantum circuit architecture, for a quantum computer, based on a point-symmetry group of a physical system, the method comprising:
receiving the point-symmetry group, wherein the point-symmetry group comprises a plurality of elements, each element corresponding to a symmetry operation on all quantum basis states of the physical system;
receiving a unitary operator based on a plurality of parameters, wherein the unitary operator encodes a quantum circuit architecture;
determining a symmetrized-unitary operator based on the unitary operator, wherein the symmetrized-unitary operator:
transforms as the identity representation of the point-symmetry group;
is based on a proper subset only of the plurality of parameters; and
encodes the compressed quantum circuit architecture; and
transmitting the symmetrized-unitary operator to a quantum computer to enable configuration of the compressed quantum circuit architecture and application of the compressed quantum circuit architecture to a quantum memory containing a first quantum basis state of the physical system stored in a plurality of qubits, wherein the first quantum basis state transforms according to a first irreducible representation of the point-symmetry group.

15. A computer program product including one or more sequences of one or more instructions which, when executed by one or more processors, cause an apparatus to at least perform the steps of the method of claim 14.
